(19) United States
(12) Patent Application Publication
(10) Pub. No.: US 2024/0124657 A1
(43) Pub. Date: Apr. 18, 2024

(54) METHOD FOR OPTIMIZATION AND RECOVERY OF SECOND-GENERATION SUGAR DILUTED STREAM AND USES THEREOF

(71) Applicants: PETRÓLEO BRASILEIRO S.A. - PETROBRAS, Rio de Janeiro (BR); CNPEM - CENTRO NACIONAL DE PESQUISA EM ENERGIA E MATERIAIS, Campinas (BR)

(72) Inventors: Luiz Fernando MARTINS BANDEIRA, Rio de Janeiro (BR); Viviane MARCOS NASCIMENTO VICENTE, Rio de Janeiro (BR); Carlos Eduardo DRIEMEIER, Rio de Janeiro (BR); Christian ALEJANDRO QUEIPO, Rio de Janeiro (BR); Danuza NOGUEIRA MOYSES, Rio de Janeiro (BR); Absai DA CONCEIÇAO GOMES, Rio de Janeiro (BR); Isabelle LOBO DE MESQUITA SAMPAIO, Rio de Janeiro (BR); Ana Paula RODRIGUES TORRES, Rio de Janeiro (BR); Felipe DE OLIVEIRA BRITO, Rio de Janeiro (BR); Adriano DO COUTO FRAGA, Rio de Janeiro (BR); Tassia LOPES JUNQUEIRA, Rio de Janeiro (BR); Aline MACHADO DE CASTRO, Rio de Janeiro (BR)

(21) Appl. No.: 18/486,650

(22) Filed: Oct. 13, 2023

(30) Foreign Application Priority Data
Oct. 14, 2022 (BR) 10 2022 0209162

(51) Int. Cl. C08H 8/00; B01D 61/58; C12P 19/04; C12P 19/14

(52) U.S. Cl. CPC C08H 8/00; B01D 61/58; C12P 19/04; C12P 19/14; B01D 61/147

(57) ABSTRACT

The present invention relates to a method for optimization and recovery of second-generation sugar diluted stream, comprising pretreatment, enzymatic hydrolysis and mainly washing the residual solid from the enzymatic hydrolysis, which allows the increase of sugar recovery. The use of this sugar diluted stream is crucial for process integration, may have different possibilities of use, for example, can be applied in the mechanical refining step, microorganisms propagation, enzyme production, fermentation, enzymatic hydrolysis, including combination of uses.

### FIELD OF THE INVENTION

The present invention falls within the field of Renewable Energy, more precisely in the area of Biomass, and refers to a method for optimization and recovery of the second-generation sugar diluted stream. The recovered and optimized diluted stream can be applied in the mechanical refining step, microorganism propagation, enzyme production, fermentation, enzymatic hydrolysis, including combination of uses.

### BACKGROUND OF THE INVENTION

The second-generation sugar production process involves carrying out two sequential steps: biomass pre-treatment (PT) and enzymatic hydrolysis (HE), there may also be a mechanical refining step between them.

The pre-treatment step (PT) aims to opening the biomass structure to expose the fraction of polysaccharides from biomass, with consequent release of sugars, especially those from the hemicellulose fraction. A pre-treatment efficiency depends on the conditions of temperature, reactor residence time and pH, which can be adjusted, for example, by the addition of acid. Several types of pretreatment (PT) that can be classified as biological (use of microorganisms), physical (grinding, mechanical refining, temperature and pressure), chemical (use of alkalis, acids or solvents) and physicochemical (as in the mentioned example, conjugation of acid or alkalis with physical agents) exist.

The enzymatic hydrolysis (HE) step consists in the digestion of pre-treated biomass, with the addition mainly from cellulolytic enzymes and resulting in release of sugars, mostly glucose from the cellulosic fraction. This step has a relevant contribution to the cost of the process, not only for the enzymes, but also due the residence time of 48 to 120 hours in agitated reactors.

At the end of enzymatic hydrolysis (HE), the mixture is directed to a solid-liquid separation step using, for example, a filter press (with the option of use of water to wash solids), obtaining two streams: a fibrous stream with not digested biomass (residue of enzymatic hydrolysis) by the enzyme and a liquid stream rich in sugars. This last stream is directed to the fermentation step by microorganisms to be converted into products with greater added value or of interest, including, but not limited to, biofuels such as second-generation ethanol.

The solid washing step was not carried out, which resulted in the loss of up to 25% of the sugar produced in biomass pre-treatment and enzymatic hydrolysis. Washing the solid allows for an increase in the recovery of sugars, but results in a sugar dilute liquid stream. The direction of this stream towards the fermentation, without a concentration step, would result in dilution of sugars and consequently, obtaining products in lower concentrations, reducing energy efficiency of the process.

To optimize sugar recovery, it is necessary to wash the solid, which results in a sugar diluted liquid stream. Failure to wash the solid implies losses of, typically, but not limited to, 10% to 25% of free sugars available after the enzymatic hydrolysis (HE) step. The use of this sugar diluted stream is crucial for process integration, may have different possibilities of use, such as, for example, return to the HE step, be a source of carbohydrate for the microorganism propagation step or have as destination a sugar concentration process, for example, by membrane separation. This process makes it possible to obtain the concentrate stream, which can be directed to the microorganism fermentation or propagation step, and the permeate stream, which can be used to cool mechanical refining equipment, whether or not associated with pre-treatment (PT). The diluted stream, as it has a lower concentration of inhibitors, whether it is concentrated by membranes or not, also becomes an interesting carbon source for the microbial growth, not only for the spread of fermentative microorganisms, but also for the production of enzymes in the in situ model.

### STATE OF THE ART

Some documents from the state of the art propose procedures for recovering lignocellulosic material, including mechanical refining and/or fermentation and its use in enzymatic hydrolysis, for example, as follow.

Document BR 112012010808 B1 relates to a process for preparing a fermentation product from lignocellulosic material comprising the following steps: a) optionally, pre-treatment; b) optionally, washing; c) enzymatic hydrolysis; d) fermentation; and e) optionally, recovery of a fermentation product. Furthermore, said document says that the product from hydrolysis step (c) is subject to solid-liquid separation and the liquid fraction is fed to an ultrafiltration unit, where the filtrate is fed to the fermentation step (d) and the retentate is recycled to step (c).

Document BR 122016020119 B1 relates to a process for preparing a fermentation product from lignocellulosic material comprising the following steps: a) optionally, pre-treatment of the lignocellulosic material; b) optionally, washing the optionally pre-treated lignocellulosic material; c) enzymatic hydrolysis of lignocellulosic material optionally washed and/or optionally pre-treated with the use of an enzyme composition comprising at least two cellulases and so that the enzyme composition at least comprises GH61; and d) optionally, recovery of a sugar product.

In this sense, both BR 112012010808 B1 and BR 122016020119 B1 describe processes for preparing a fermentation product of lignocellulosic material comprising an optional step of washing the pre-treated material for recovering sugars. It should be noted that, even in these cases, there will be a residual of sugars not used in the enzymatic hydrolysis step that will be lost if a diluted sugar stream is not recovered from the solid remaining from hydrolysis enzymatic. Therefore, for a process without this separation of streams, a technical solution is needed that allows greater recovery of sugars in order to return them to the process, reducing losses.

Document U.S. Pat. No. 9,580,454 B2 relates to a fractionation process to produce products from a lignocellulosic biomass comprising the following steps: i) chemical treatment to reduce chemical bonds between lignin and carbohydrates from said lignocellulosic biomass, said chemical treatment being carried out before mechanically refining in step (ii), ii) mechanically refining lignocellulosic biomass comprising said lignin and carbohydrates from step (i) in a mechanical refiner under refining conditions to disintegrate and reduce particle size of lignocellulosic biomass and form a refined biomass of said lignin and carbohydrates, iii) enzymatic hydrolysis of carbohydrates in the refined biomass into sugars to form a mass comprising a fraction of sugar and a fraction of lignin, iv) separating the lignin fraction from the sugar fraction, v) recovering said lignin fraction and recovering said sugar fraction, and vi) fermentation of said sugar fraction to form sugar bioproducts selected from biofuels, sugar alcohols and sugar acids as products.

The document in the name of Dou et al., entitled “Post-treatment mechanical refining as a method to improve overall sugar recovery of steam pre-treated hybrid poplar”, relates to a study of mechanical refining to improve the sugar yield from biomass processed under a wide range of steam pre-treatment conditions. The resulting water-insoluble fractions were subjected to mechanical refining. After refining, the pre-treated poplar obtained a 32% improvement in enzymatic hydrolysis and achieved general monomeric sugar with similar recovery (539 kg/ton) to that obtained for samples pre-treated with SO₂. This document demonstrates the possibility of using post-treatment refining to accommodate possible disorders of the pre-treatment process without sacrificing the production of sugar. Refining demonstrated a method to partially decouple pre-treatment with enzymatic hydrolysis and fermentation. These findings illustrate the potential of refining to minimize the impact of fluctuations of pre-treatment process and increase the stability of future biorefineries.

The document in the name of Jones et al., entitled “Enhancement in enzymatic hydrolysis by mechanical refining for pre-treated hardwood lignocellulosics”, relates to a study on the effectiveness of mechanical refining to overcome biomass recalcitrance barrier. Refining at laboratory scale was carried out using PFI mills and valley beaters refiners using green liquor and hardwood kraft pulps. A strong positive correlation was determined between sugar recovery and the value of water retention. Refining produced significant improvements in the enzymatic hydrolysis yield in relation to unrefined substrates (e.g., the recovery of sugar increased from 67% to 90%). An absolute maximum improvement of enzymatic hydrolysis with refining was observed in enzymatic hydrolysis conditions that produced intermediate conversion levels. For target sugar conversion of 91%, PFI refining at 4000 revolutions allowed a reduction of 32% in enzyme load to 15% hardwood cellulose with lignin content and 96 h of hydrolysis time, in comparison with unrefined material.

The above-described prior art documents, in its turn, describe mechanical refining processes of the lignocellulosic material. It is a fact that mechanical refining can improve the digestibility of the material in the enzymatic hydrolysis step, increasing the availability of fermentable sugars in the liquor. However, even with refining, the enzymatic hydrolysis step generates a solid residue that includes a remainder of unused sugars in later steps of the process, so that their recovery in the form of a diluted sugar stream allows the advantages already explained above. It is made reference to traditional fermentable sugar recovery in the liquid fraction arising from enzymatic hydrolysis, however, in none of these documents there is a mention of such sugar recovery from the remaining solid residue.

As can be seen, although there is, in the state of the art, solutions to minimize problems found for recovery of lignocellulosic material, no documents were observed that could anticipate in its entirety the present invention, nor make the achieved solution obvious by the present invention, to propose a method for optimization and recovery of the diluted sugar stream after washing the solid (enzymatic hydrolysis residue) and possibility of use in the mechanical refining step, microorganism propagation, enzyme production, fermentation, enzymatic hydrolysis, including combination of uses.

Furthermore, the present invention, in addition to provide suitable solutions for the use of diluted sugar stream resulting from washing pre-treated biomass undigested by the enzyme, also provides the solution for another difficulty identified during the fiber mechanical refining step, which is the need to add water, whether for sealing or cooling the discs for operation of the equipment. The present invention employs the sugar diluted stream after washing the enzymatic hydrolysis residue or the permeate stream of the membrane separation system, allowing the integration of stream and reduction of process water consumption.

### SUMMARY OF THE INVENTION

The present invention aims to propose a method for optimization and recovery of second-generation sugar diluted stream mainly comprising washing the solid residue from enzymatic hydrolysis, which enables increased sugar recovery. The use of this diluted sugar stream is crucial for process integration, which may have different possibilities of use, for example, being able to be applied in mechanical refining step, microorganism propagation, enzyme production, fermentation, enzymatic hydrolysis, including combination of uses.

### BRIEF DESCRIPTION OF THE FIGURES

To obtain a full and complete view of the objective of this invention, the figure is presented to which reference is made, as follows.

FIG. 1 shows a representation of the process involved in the present invention.

FIG. 2 shows a schematic representation of the disc refiner.

FIG. 3 shows the appearance of the material resulting from the mechanical refining process of pre-treated material, with photographs referring to the pre-treated material before (PT) and after the process (PT+RM).

FIG. 4 shows the appearance of the acid pretreated material before (PT) and after mechanical refining (PT+RM) dispersed in water.

### DETAILED DESCRIPTION OF THE INVENTION

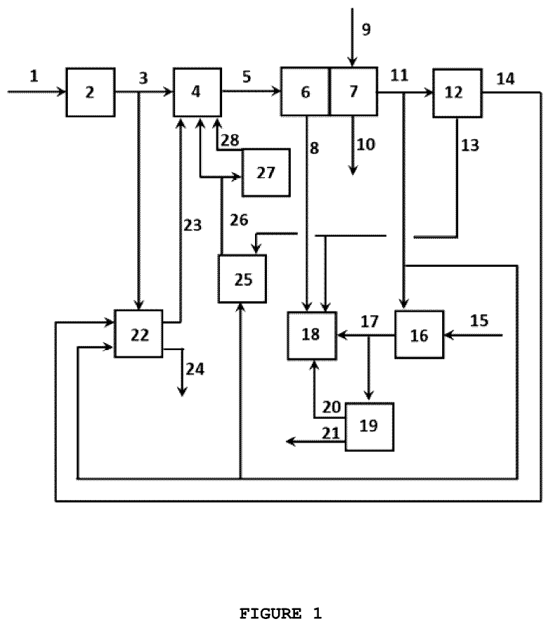

The present invention relates to a method for optimization and recovery of the second-generation diluted sugar stream obtained from the solid residue from enzymatic hydrolysis. FIG. 1 shows a representation of the entire process related to the present invention.

Initially, the biomass (1) is subjected to pre-treatment step (2) aiming to open the biomass structure, exposure of polysaccharides and release of some second-generation sugars, in oligomeric or monomeric form, mainly xylose and glucose. Pre-treated biomass (3) can be sent to the enzymatic hydrolysis step (4) or to mechanical refining (22).

In enzymatic hydrolysis (4) digestion occurs of previously pre-treated biomass, with the production of sugars, mainly in monomeric form and, especially glucose. Preferably after 48 to 96 hours, the hydrolyzate (5) is destined for solid-liquid separation (6 and 7), more specifically, but not limited to, filter presses, where a liquid stream (8), rich in sugars, which is directed to the fermentation step (18) by microorganisms to be converted into products with higher added value or interest, including but not limited to, biofuels such as second-generation ethanol, and a solid stream (cake).

Therefore, the method proposed in the present invention aims to increase the recovery of sugars retained in fibrous chain (cake) with biomass not digested by the enzyme, and comprises the following steps:
(a) adding water to the solid residue (cake) remaining from enzymatic hydrolysis; and
(b) collecting a sugar diluted liquid stream (11) and a washed solid stream (10).

In step (a), the addition of water must occur preferably integrated into the primary solid-liquid separation method, using, for example, a filter press with cake washing technology, and must start after recovery of the concentrated stream rich in sugars. By starting adding water, the remaining water in the cake represents 40 to 70% of the cake mass. The proportion of water added to the cake is 1 to 3 m³ of water per ton of total solids, being conducted between 50° C. and 75° C.

The cake washing configuration, e.g., in a filter press, must favor the washing mechanism by displacement to the detriment of the washing mechanism by dilution to reduce the volume of wash water.

The added water can be partially composed of the diluted stream collected from washing. With this recirculation, it is possible to increase the recovery of sugars with the same washing rate or, alternatively, reduce process water consumption to identical recovery, optimizing the use of water in relation to conventional washing without recirculation.

In step (b), collecting the diluted stream must occur without changing the concentrated stream previously collected. The diluted stream will have a fermentable sugars total concentration typically less than 50 g/L. The solid residue after collecting the diluted stream should preferably contain less than 5% of the total sugars produced in enzymatic hydrolysis.

The generated liquid stream (11) can be intended for at least one of the selected steps of the group comprising the microorganism propagation (16), the enzyme production (25), mechanical refining (22), fermentation, enzymatic hydrolysis and biogas production.

For example, stream (11) can be sent to the microorganism propagation (16), where the microorganism strain (15), preferably yeast, is added to the cell multiplication/growth phase. After microorganism propagation (16), the broth (17) is directed to the fermentation step (18), where the sugar conversion in other products with higher added value or of interest occurs. Alternatively, the broth (17) can be sent to centrifugation step (19), obtaining the stream (20) with a higher concentration of propagated cells and the supernatant stream (21).

The stream (11) can also be sent to enzyme production (25), in which it acts as a supplementary sugar source supply to the microorganism producer, preferably filamentous fungi. This stream also integrates part of the water demand of this step of the process due to its low concentration of inhibitors and be a solid-free stream. After the enzyme production step in situ, the medium rich in cellulases (26), preferably without solid/liquid separation or additional treatments, “whole broth” or with separation of microorganisms (27 and 28), is directed to the enzymatic hydrolysis (4).

Furthermore, the stream (11) can also be intended for mechanical refining equipment, which can require water to adjust the consistency of the biomass input and requires a stream of water to sealing/cooling the discs. Mechanical refining (22) has the objective of shearing and comminuting the biomass fiber, increasing the specific area and favoring the action of the enzyme. Around 10 to 35%, more specifically 10% to 15% of the water used to seal/cool the discs is incorporated to pre-treated and refined biomass (23). The other part of diluted sugar stream results in stream (24), with temperature from 20° C. to 60° C., more specifically 40° C., which can have the same applications as the stream (11).

Mechanical refining can be carried out additionally to the pre-treatment of biomass and aims to the comminution of the fiber, with the consequent increase in the specific area, favoring the action of the enzyme during enzymatic hydrolysis. Any biomasses can be used, such as bagasse and sugar cane straw and bagasse of pre-treated energy cane, however, not limited to them.

The definition of the stream to be used depends on tolerance to the formation of scale inside the mechanical refining equipment or microbial contamination risks for the production process since sugars are substrates for the metabolism of microorganisms. These risks are higher in equipment that operates in an intermittent way, as is the case with large-scale development pilot, being smaller in continuous operation on a commercial scale. As a precaution, heat treatments or addition of antiscales or biocides can be carried out, as long as they do not impact subsequent steps of the process.

For example, in a sugar production plant 2G or integrated 1G/2G, both for the production of biofuels or bioproducts with higher added value, different steps of the process depend on sugar streams as fundamental inputs, such as the enzyme production, microorganism propagation, and the fermentative processes themselves. These streams are supplied by diversions into the main stream of sugars generated in the plant or acquired as additional inputs. Partial or full use of the diluted stream in each of these steps, preferably not in the fermentation step, has a positive impact in operating costs.

### Examples of the Invention

In solid-liquid separation tests in filters press on a pilot scale, without washing the solid, significant losses were identified, more specifically, but not limited to, up to 25% of sugars produced in the enzymatic hydrolysis step, indicating the need to wash the cake to increase sugar recovery. The filter press used was composed of 20 boards with an area of 4.32 m² and double canvas as an filter element.

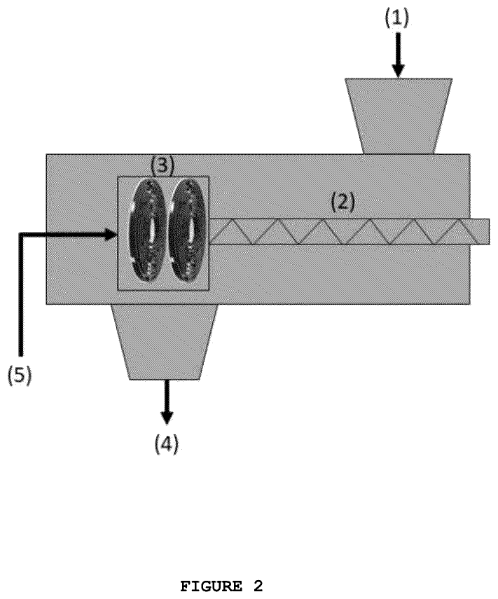

In the tests, a pilot scale mechanical refiner was used with 12-inch diameter discs. One schematic of the mechanical refining equipment is shown in FIG. 2.

In general, the refiner acts by boosting the biomass placed in its material reservoir (1) in direction to the pair of discs through a feed screw (2) with controllable speed. The refiner discs (3) promote the mechanical refining of the material, with speeds and also controllable distances. The refined material between discs leads to the refiner outlet (4), which can be collected at this point. An important operating detail of this equipment is the need for a water flow rate (5) to the cooling of the discs and for the refining itself, which translates into a refined material with moisture content higher than that of the original starting material.

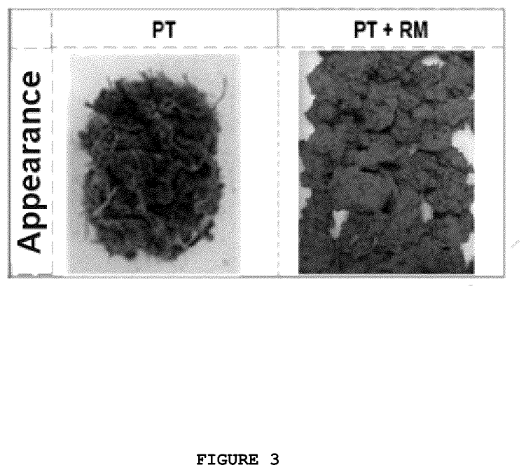

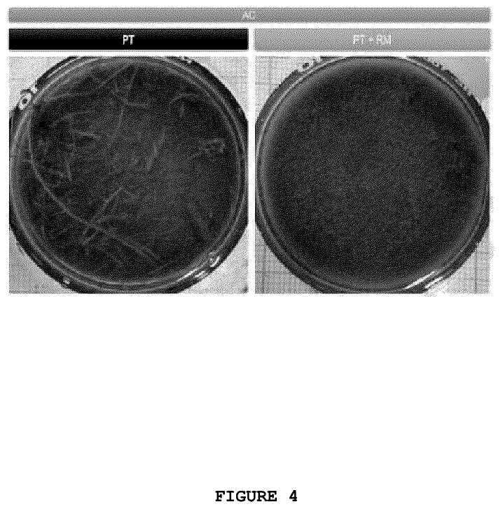

The particle size distribution of the refined material could not be evaluated given the limitations of methods available in the laboratory: classification by sieves is limited to dry materials and laser diffraction is limited to particles with a maximum size of 2 mm. In this way, evaluations were carried out regarding the appearance of the materials, in terms of morphology (crystallinity) and digestibility in enzymatic hydrolysis reactions. These last two assessments were focused on contributing to the analysis of the results of enzymatic hydrolysis, whose performance is not the object of the present invention. Regarding the appearance, in experiments conducted with sugar cane bagasse, energy cane bagasse or straw, all pre-treated biomass, reduced the content of coarse fibers and fiber sizes were reduced (FIG. 3), especially for samples after acid pre-treatment, as shown in FIG. 4.

The conditions of the fiber after the mechanical refining process depend on the rotation of the feed screw (7 to 20 rpm), which is associated with the biomass flow rate, rotation of discs (1500 to 3000 rpm) and disc spacing (1 to 10 mm). To increase the severity of mechanical refining, the spacing between discs has been reduced to as little as 0.1 mm.

Table 1 shows that the temperature of the refined material increases from 24° C. to around 40° C. due to partial transformation of the mechanical energy of the discs into thermal energy, which justifies the importance of cooling the discs with water.

TABLE 1
Summary of measured and calculated parameters in
mechanical refining tests.

| | | HT | | AC | |
|---|---|---|---|---|---|
| | | Input | Output | Input | Output |
| Measured | Wet mass (kg) | 28.65 | 41.5 | 28.70 | 42.0 |
| | Moisture (%) | 70.48 | 82.5 | 68.22 | 79.3 |
| | Time of collecting (hh:mm) | 21:14 | 22:04 | 22:16 | 23:06 |
| | Water mass, output 2 (kg) | — | 11.9 | — | 11.0 |
| | Material temperature (° C.) | 24.10 | 40.1 | 24.80 | 41.3 |
| Calculated | Dry mass (kg) | 8.5 | 7.3 | 9.1 | 8.7 |
| | Time of collecting (hh:mm) | 00:50 | | 00:50 | |
| | Water mass, output 1 (kg) | — | 14.0 | — | 13.7 |
| | Water mass, total (kg) | — | 25.9 | — | 24.7 |
| | Biomass flow rate (kg/h | — | 10.1 | — | 10.9 |
| | db) | | | | |
| | Water flow rate, total | — | 31.1 | — | 29.7 |
| | (kg/h) | | | | |
| | Water flow rate-output 1 (kg/h) | | 16.9 | | 16.5 |
| | Water flow rate-output 2 (kg/h) | | 14.3 | | 13.2 |
| | Consumed energy (kWh/ton | 237 | | 338 | |
| | of pre-treated bagasse) | | | | |

Table 1 also shows the water flow rates of 31.1 kg/h for hydrothermally pretreated (HT) material and 29.7 kg/h for the material pre-treated with acid (AC), which represents a ratio greater than 3 kg of water/kg of dry pre-treated biomass fed to the mechanical refiner. In view of this high ratio in the mechanical refining operation, this innovation sought to reduce the need of new water to the process.

Additionally, tests conducted in mechanical refining equipment indicated the incorporation of 10 to 35% of water used for sealing/cooling the discs in the pre-treated and refined biomass, causing dilution of this stream and damaging the energy efficiency of the process.

Because the equipment is on a pilot scale and with limitations of lines and process steps, new water was always injected into the equipment. For a process continuously operating and on a larger scale, the consumption of new water would be economically unviable, with the need to have a closed circuit and the use of more appropriate streams of the production process. In this way, the innovation suggested in the present invention proposes the use of the sugar diluted stream instead of pure water, which will lead to greater integration of process streams and energy efficiency.

In this way, the invention shows solutions for the use of the diluted sugar stream after washing the solid (enzymatic hydrolysis residue), proposing its use in the mechanical refining step, microorganism propagation, enzyme production, fermentation, enzymatic hydrolysis, including combination of uses.

### Application of the Invention

The method proposed in the present invention can be applied in different second-generation sugar production processes, which adopt a solid-liquid separation step for removing undigested fiber after enzymatic hydrolysis. The invention is applicable to different models of mechanical refining equipment, in some cases by mechanical refining equipment manufacturer restriction for the sealing or cooling water of the discs, the stream (11) can be sent to a separation system by membranes (12), optionally composed of a pre-filter, microfiltration and reverse osmosis, resulting in two streams, one being called retentate (13), rich in sugar and sent, for example, to the sugar fermentation step (18) or enzyme production (25) and other stream called permeate (14), intended for the mechanical refining equipment (22).

The diluted stream (11) can also be intended for the enzymatic hydrolysis step itself, composing part or all of the water stream added to this step, returning the sugars in the process. The concentration of sugars in the reactor, at the beginning of the process, with the entry of part of this stream did not affect the performance of the cellulolytic enzymes.

The stream (11) can be used for production of biogas, through anaerobic digestion, which could produce up to 350 NmL of CH₄/g COD consumed depending on stream nutrient conditions, such as sources of N and P.

For example, in enzymatic hydrolysis processes with low solids content, the water stream added to the process becomes a relevant stream, at the same time whereas processes with high solids content, the additional water stream would correspond to less than 30% of the solid washing stream (diluted stream). The diluted stream when used in mechanical refining, around 65% to 90% of the stream becomes available for other uses. A diluted stream, with the characteristics indicated in the present invention, has a low concentration of inhibitors, which makes it attractive to be used as a carbon source in any of the steps in which sugars are inputs, reducing process costs.

Preferably in steps such as the enzyme production and yeast propagation, in which sugars acquired products necessarily undergo dilution and are water intensive steps, the diluted stream would represent a small part of the reaction volume, and the choice of one or another step will depend on the assessment of the step in which the replacing inputs with this stream will bring more economic benefits to the process, since the microorganisms used in both steps are capable of use both sugars present in this diluted stream.

### Advantages of the Invention

### Economic/Productivity

The economic advantage is due to the greater use of sugars from the production process by employing the diluted stream in steps that do not require high concentration of sugars (mechanical refining, microorganism propagation, enzyme production, fermentation, enzymatic hydrolysis, including the combination of uses), avoiding the acquisition of input such as molasses (concentrated sugar stream obtained in the sugar production process), with higher cost and incidence of taxes. By increasing the use of sugars also have an environmental advantage, as the product generation without additional inputs. Additionally, by employing stream as a utility for mechanical refining, water capture is reduced since there is process stream usefulness.

1. A method for optimization and recovery of a second generation sugar diluted stream comprising:
(a) adding water to a remaining solid residue (cake) from enzymatic hydrolysis; and
(b) collecting a sugar diluted liquid stream and a washed solid stream.

2. The method according to claim 1, wherein when starting to add water, the water remaining in the cake represents 40 to 70% of the mass of the cake.

3. The method according to claim 1, wherein a ratio of water added to the cake is 1 to 3 m³ of water per ton of total solids, and wherein the addition of water is conducted between 50° C. to 75° C.

4. The method according to claim 1, wherein the diluted liquid stream is passed through a membrane, followed by collecting the retentate and the permeate.

5. The method according to claim 1, wherein the collected diluted stream presents a total concentration of fermentable sugars less than 50 g/L.

6. The method according to claim 1, wherein the solid residue after collection of the diluted stream contains less than 5% of the total sugars produced in enzymatic hydrolysis.

7. A method of using the diluted stream resulting from the method of claim 1 for one or more of propagation of microorganisms, production of enzymes, mechanical refining, fermentation, enzymatic hydrolysis and biogas production.

8. A method of using the diluted stream resulting from the method of claim 7, wherein the retentate is used for fermentation of sugars and the permeate is used for mechanical refining.
